(19) United States
(12) Patent Application Publication
(10) Pub. No.: US 2005/0037451 A1
(43) Pub. Date: Feb. 17, 2005

(54) Method of using potassium permanganate in water analysis

(76) Inventors: Bo-Cun Chen, Sansia Township (TW); Chiao-Chung Huang, Dasi Township (TW); Ching-Wei Liao, Taipei City (TW); Guo-Ming Huang, Taichung City (TW)

Correspondence Address: RABIN & Berdo, PC, 1101 14TH STREET, NW, SUITE 500, WASHINGTON, DC, 20005, US

(21) Appl. No.: 10/814,233

(22) Filed: Apr. 1, 2004

(30) Foreign Application Priority Data
Aug. 14, 2003 (TW) 92122431

(51) Int. Cl. C12Q 1/04

(52) U.S. Cl. 435/34

(57) ABSTRACT

A method of water analysis for detecting the presence of microorganisms in a water sample, comprising the steps of: first, providing a bio-membrane as a filter; filtering out the microorganisms in the water sample, using the bio-membrane; cultivating the microorganisms on the bio-membrane; staining the microorganisms on the bio-membrane with potassium permanganate (KMnO₄); rinsing the bio-membrane with purified deionized water; and finally, counting microorganisms.

This application claims the benefit of Taiwan application Serial No. 92122431, filed Aug. 14, 2003.

### BACKGROUND OF THE INVENTION

1. Field of the Invention

The invention relates in general to a method of water analysis, and more particularly to a method of water analysis for detecting the presence of microorganisms in a water sample, including the step of staining the microorganisms with potassium permanganate.

2. Description of the Related Art

There are various complicated processes and impressionable procedures for manufacturing all kinds of devices in semiconductor industry. One important step of these is cleanliness of a wafer by using deionized water in preventing the contaminants on the surface of the wafer during the manufacturing processes, such as dust. In order to use water as industrial water it is often necessary to free it of impurities or to determine the amount of impurities in an aqueous solution. Successful water analysis helps in monitoring and controlling quality of deionized water used in cleaning the wafer so that the accuracy and precision of the products can be controlled well.

Referring to FIG. 1, a flow chart showing a conventional method of water analysis, includes steps 101, 102, 103, 104A, 104B, 104C, 104D, 105A, 105B, 105C, 105D, 106A, 106B, 106C, and 106D. Each essay is performed in triplicate. In the step 101, four bio-membranes 1a, 1b, 1c and 1d are provided and the pore size of the bio-membranes 1a, 1b, 1c, 1d is about 0.3 μm in diameter. Next, in the step 102, four water samples, one of which with 100 milliliters (ml), are provided, each water sample is filtered through a corresponding bio-membrane 1a, 1b, 1c, 1d, respectively, with the aid of a vacuum filtration technique. The microorganisms are thus trapped by the bio-membranes. Then, in the step 103, microorganisms on the bio-membranes 1a, 1b, 1c, 1d are cultivated at about 30° C. with 2 ml of nutrient solution on each bio-membrane.

The microorganisms trapped on different bio-membranes are cultivated for different time period. For example, the microorganisms on the bio-membrane 1a is cultivated for 24 hours (hrs), in the step 104A; the microorganisms on the bio-membrane 1b is cultivated for 48 hours, in the step 104B; the microorganisms on the bio-membrane 1c is cultivated for 72 hours, in the step 104C; the microorganisms on the bio-membrane 1d is cultivated for 96 hours, in the step 104D.

At the end, in the step 105A, 105B, 105C, 105D, take count of microorganism colonies on the bio-membranes 1a, 1b, 1c, and 1d respectively, under a microscope. Microorganism population is determined according to amounts of readable microorganism colonies. Also, in the step 106A, 106B, 106C, 106D, take photographs under the microscope, and then FIG. 3A, FIG. 3B, FIG. 3C, and FIG. 3D are obtained as micrographs of the bio-membrane cultivated for 24 hours, 48 hours, 72 hours and 96 hours, respectively.

However, one may notice that the microorganisms on the bio-membranes 1a, 1b, 1c, 1d are indistinct and difficult to be identified when the microorganisms cultivated for different time period are directly examined under the microscopy, as shown in FIG. 3A, 3B, 3C and 3D. In particular, the microorganisms are very tiny so that it is difficult to identify and determine current amounts of readable microorganism colonies when the microorganism colonies are aggregate on the bio-membranes 1a, 1b, 1c, and 1d. Therefore, it is necessary to provide a method for easily detecting the presence of microorganisms in a water sample in order to shorten the time of the semiconductor manufacturing processes.

### SUMMARY OF THE INVENTION

In view of the foregoing, it is therefore a method of water analysis of the present invention is provided for easily detecting the presence of microorganisms in a water sample. The invention also improving microorganism discrimination by staining the microorganisms with potassium permanganate (KMnO₄).

The invention achieves the above-identified objective by providing a method of water analysis, for detecting the presence of microorganisms in a water sample, including the steps of (a) providing a bio-membrane as a filter; (b) filtering out the microorganisms in a water sample, using the bio-membrane; (c) cultivating the microorganisms on the bio-membrane; (d) staining the microorganisms on the bio-membrane with potassium permanganate (KMnO₄); (e) rinsing the bio-membrane with purified deionized water; and (f) counting microorganisms.

Other objects, features, and advantages of the invention will become apparent from the following detailed description of the preferred but non-limiting embodiments. The following description is made with reference to the accompanying drawings.

### BRIEF DESCRIPTION OF THE DRAWINGS

FIG. 1 (Prior Art) is a flow chart showing a conventional method of water analysis.

FIG. 2 is a flow chart showing the method of water analysis in accordance with a preferred embodiment of the invention.

FIG. 3A is a micrograph of the bio-membrane cultivated for 24 hours in accordance with the conventional method of water analysis in FIG. 1.

FIG. 3B is a micrograph of the bio-membrane cultivated for 48 hours in accordance with the conventional method of water analysis in FIG. 1.

FIG. 3C is a micrograph of the bio-membrane cultivated for 72 hours in accordance with the conventional method of water analysis in FIG. 1.

FIG. 3D is a micrograph of the bio-membrane cultivated for 96 hours in accordance with the conventional method of water analysis in FIG. 1.

FIG. 4A is a micrograph of the bio-membrane cultivated for 24 hours in accordance with the preferred embodiment of the invention in FIG. 2.

FIG. 4B is a micrograph of the bio-membrane cultivated for 48 hours in accordance with the preferred embodiment of the invention in FIG. 2.

FIG. 4C is a micrograph of the bio-membrane cultivated for 72 hours in accordance with the preferred embodiment of the invention in FIG. 2.

FIG. 4D is a micrograph of the bio-membrane cultivated for 96 hours in accordance with the preferred embodiment of the invention in FIG. 2.

FIG. 5, a diagram of identify rate vs. time (days after cultivation) curves. The values shown were mean value of triplicate.

FIG. 6, a micrograph of the bio-membrane in accordance with the invention, shows that the maximum readable microorganism colonies stained by the method of the invention is about 184.43 μm in diameter when seen through the microscope, of which the power of magnification is 500×.

FIG. 7, a micrograph of the bio-membrane in accordance with the invention, shows that the minimum readable microorganisms stained by the method of the invention is about 39.10 μm in diameter when seen through the microscope, of which the power of magnification is 1000×.

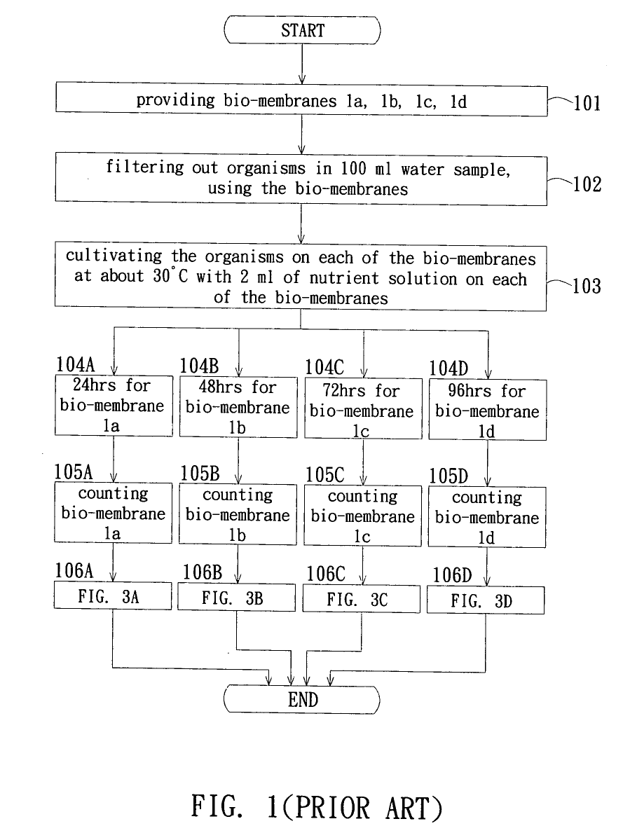

### DETAILED DESCRIPTION OF THE INVENTION

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like components throughout.

Potassium permanganate (KMnO₄) is a dark purple crystalline compound, used as an oxidizing agent and disinfectant and in deodorizers and dyes. One of the characteristics of the present invention is that the microorganism colonies in the water sample are stained with 0.02 M potassium permanganate. As a result, the dyed microorganism colonies on the bio-membranes become dark brown and can therefore be easily identified. In spite of potassium permanganate, the strong oxidizer, kills all dyed microorganisms, the current amounts of the microorganism colonies can still be determined straightforward.

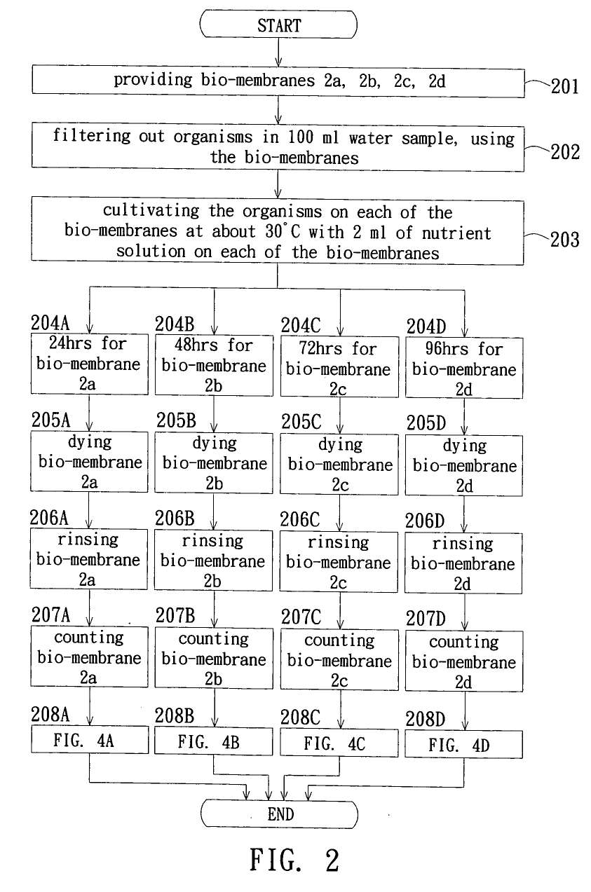

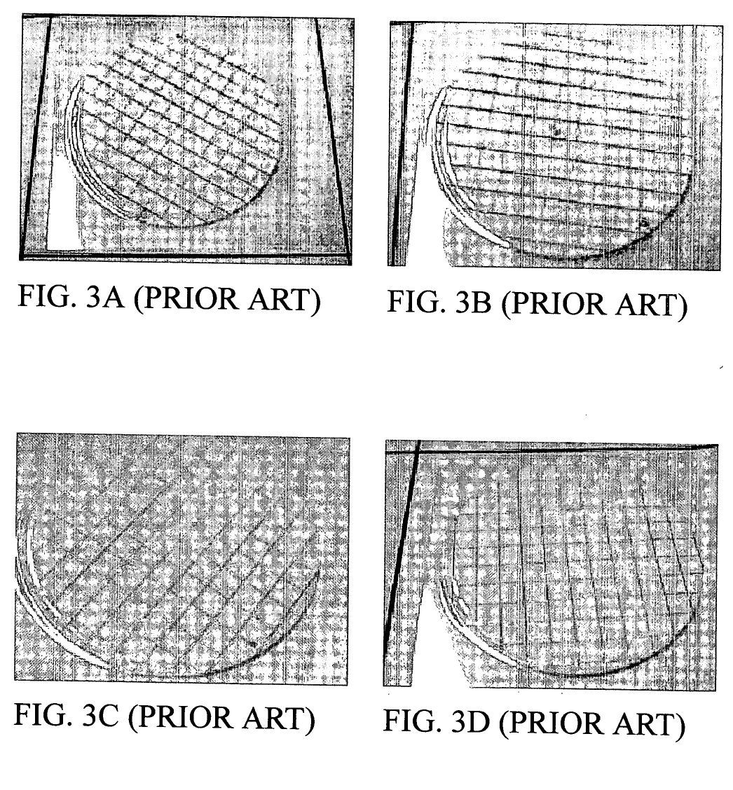

Referring to FIG. 2, a flow chart showing the method of water analysis in accordance with a preferred embodiment of the invention, includes steps 201, 202, 203, 204A, 204B, 204C, 204D, 205A, 205B, 205C, 205D, 206A, 206B, 206C, 206D, 207A, 207B, 207C, 207D, 208A, 208B, 208C, and 208D. Each essay is performed in triplicate. In the step 201, four bio-membranes 2a, 2b, 2c and 2d are provided and the pore size of each of the bio-membranes 2a, 2b, 2c, 2d is about 0.3 μm. Next, in the step 202, four water samples, one of which with 100 milliliters (ml), are provided, each water sample is filtered through a corresponding bio-membranes 2a, 2b, 2c, 2d, respectively, preferably with the aid of the vacuum filtration technique. The microorganisms are thus trapped by the bio-membranes. Then, in the step 203, microorganisms on the bio-membranes 2a, 2b, 2c, 2d are cultivated at about 30° C. with 2 ml of nutrient solution on each bio-membrane.

The microorganisms trapped on different bio-membranes are cultivated for different time period. For example, the microorganisms on the bio-membrane 2a is cultivated for 24 hours (hrs), in the step 204A; the microorganisms on the bio-membrane 2b is cultivated for 48 hours, in the step 204B; the microorganisms on the bio-membrane 2c is cultivated for 72 hours, in the step 204C; and the microorganisms on the bio-membrane 2d is cultivated for 96 hours, in the step 204D.

Further, in the steps 205A, 205B, 205C, and 205D, the microorganisms on the bio-membranes 2a, 2b, 2c, 2d are separately stained by using potassium permanganate (KMnO₄), with a concentration of 0.02 M (mole per liter), preferably for about 10 to 30 seconds. Next, in the steps 206A, 206B, 206C, and 206D, the bio-membranes 2a, 2b, 2c, 2d are rinsed by using purified deionized water to wash KMnO₄ out.

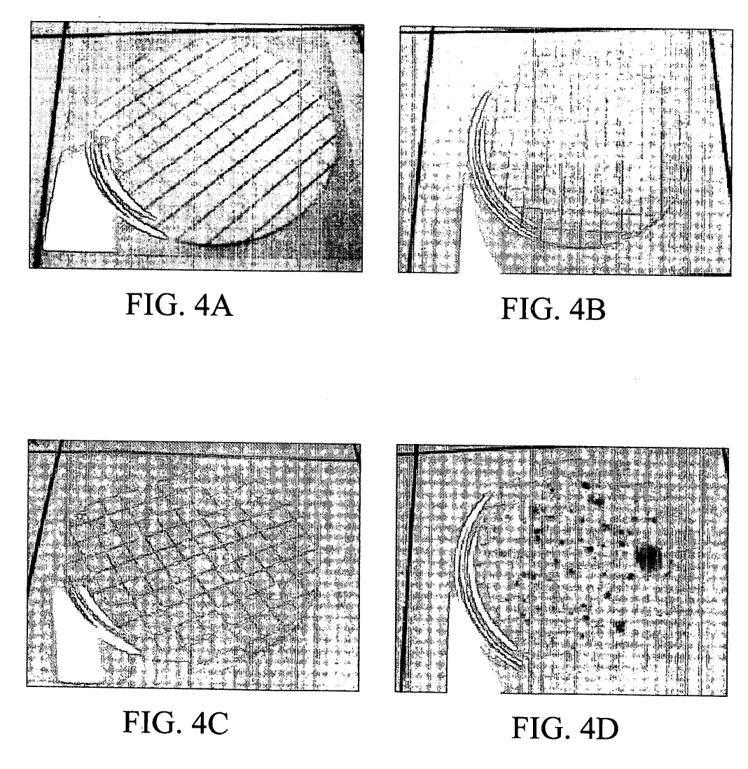

At the end, in the step 207A, 207B, 207C, 207D, take count of the microorganism colonies on the bio-membranes 2a, 2b, 2c, and 2d respectively, under a microscope. Microorganism population is determined according to amounts of readable microorganism colonies. Also, in the step 208A, 208B, 208C, 208D, take photographs under the microscope, and then FIG. 4A, FIG. 4B, FIG. 4C and FIG. 4D are obtained as micrographs of the bio-membrane cultivated for 24 hours, 48 hours, 72 hours and 96 hours, respectively.

Table 1 is a list of two experiment results of the conventional methods and of the method of the invention.

TABLE 1

| | readable | | | | |
|---|---|---|---|---|---|
| | microorganism | 1a | 1b | 1c | 1d |
| RESULTS | colony no. vs. time | (24 hrs) | (48 hrs) | (72 hrs) | (96 hrs) |
| conventional | readable | 8, | 26, | 50, | 50, |
| method in | microorganism | 11, | 30, | 52, | 54, |
| | colony no. | 14 | 28 | 49 | 56 |
| | average no. of | 11 | 28 | 50 | 53 |
| | readable micro- | | | | |
| | organism colonies | | | | |
| | identify rate (%) | 20.75 | 52.83 | 94 | 100 |
| method of | readable dye | 41, | 47, | 51, | 52, |
| the present | microorganism | 39, | 52, | 53, | 56, |
| invention in | colony no. | 36 | 48 | 54 | 58 |
| | average no. of | 39 | 49 | 53 | 55 |
| | readable dyed | | | | |
| | microorganism | | | | |
| | colonies | | | | |
| | identify rate (%) | 70.91 | 89.09 | 96.36 | 100 |

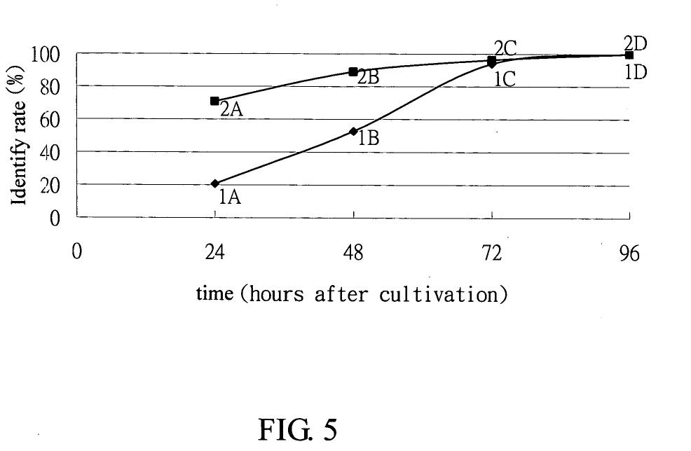

Further, a diagram of identify rate vs. time (hours after cultivation) curves is achieved as FIG. 5 in accordance with the data in Table 1. According to the result shown in FIG. 5, assumed that trapped microorganisms grow up to their maximum no. after cultivated for 96 hours, and the identify rates of the bio-membrane 1d, 2d are both defined as 100%. Then the identify rates of the bio-membrane 1a, 1b, 1c are obtained by dividing the average no. of readable microorganism colonies on the bio-membrane 1a, 1b, 1c by the average no. of readable microorganism colonies on the bio-membrane 1d, respectively. Also, the identify rates of the bio-membrane 2a, 2b, 2c are obtained by dividing the average no. of readable dyed microorganism colonies on the bio-membrane 2a, 2b, 2c by the average no. of readable dyed microorganism colonies on the bio-membrane 2d, respectively. In Table 1 and FIG. 5, the identify rate of the bio-membrane 1b cultivated for 48 hours is about 53%, but the identify rate of the bio-membrane 2b cultivated for 48 hours is about 89%. The identify rate of the bio-membrane 2b cultivated for 48 hours has increased by about 37% as compared with the identify rate of the bio-membrane 1b cultivated for 48 hours. Thus, the method of using potassium permanganate in water analysis according to the present invention can efficiently reduces the time that allow about 90% identify rate to be obtained.

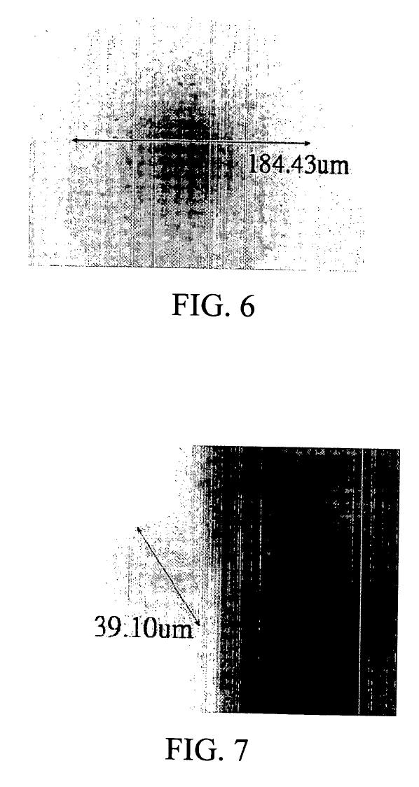

FIG. 6 and FIG. 7 are two micrographs of the bio-membrane in accordance with the invention. It is shown in FIG. 6 that the maximum readable microorganism colonies dyed by the method of the invention is about 184.43 μm in diameter when seen through the microscope, of which the power of magnification is 500×. In addition, the minimum readable microorganisms dyed by the method of the invention are about 39.10 μm in diameter when seen through the microscope, of which the power of magnification is 1000×, as shown in FIG. 7. Thus it is vary clear that the present invention can easily detect the presence of microorganisms in a water sample during the semiconductor manufacturing processes, using potassium permanganate as dyes.

In summary, the present method of using potassium permanganate in water analysis possesses the advantages of time-saving and ease for detecting the presence of microorganisms in a water sample during the semiconductor manufacturing processes compared with the conventional method. Also, the present method is an economic method for identifying the microorganism colonies because of the low prices of potassium permanganate.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A method of water analysis for detecting the presence of microorganisms in a water sample, comprising the step of staining the microorganisms with potassium permanganate (KMnO₄).

2. A method of water analysis for detecting the presence of microorganisms in a water sample, comprising the steps of:
providing a bio-membrane as a filter;
filtering out the microorganisms in the water sample, using the bio-membrane;
cultivating the microorganisms on the bio-membrane;
staining the microorganisms on the bio-membrane with potassium permanganate (KMnO₄);
rinsing the bio-membrane with purified deionized water; and
performing a colony count for readable microorganisms on the bio-membrane.

3. The method of water analysis according to claim 2, wherein the pore size of the bio-membrane is about 0.3 μm in diameter.

4. The method of water analysis according to claim 2, wherein the water sample is filtered through the bio-membrane by a vacuum filtration technique.

5. The method of water analysis according to claim 2, wherein the microorganisms are cultivated on the bio-membrane at about 30° C., using 2 ml of nutrient solution.

6. The method of water analysis according to claim 2, wherein the concentration of KMnO₄ is about 0.02 M (mole per liter).

7. The method of water analysis according to claim 2, wherein the microorganisms on the bio-membrane are stained with KMnO₄ for about 10 to 30 seconds and then the bio-membrane is rinsed with purified deionized water.

8. A method of water analysis for separately detecting the presence of microorganisms in a plurality of water samples, comprising the steps of:
providing a plurality of bio-membranes as filters;
filtering out the microorganisms in each of the water samples, using a corresponding bio-membrane, separately;
cultivating the microorganisms on different bio-membranes for different time period;
staining the microorganisms on each of the bio-membranes with potassium permanganate (KMnO₄);
rinsing each of the bio-membranes with purified deionized water; and
performing a colony count for readable microorganisms on each of the bio-membranes.

9. The method of water analysis according to claim 8, wherein the pore size of the bio-membrane is about 0.3 μm in diameter.

10. The method of water analysis according to claim 8, wherein each of the water samples is filtered through a corresponding bio-membrane by a vacuum filtration technique.

11. The method of water analysis according to claim 8, wherein the microorganisms are cultivated on each of the bio-membranes at about 30° C., using 2 ml of nutrient solution.

12. The method of water analysis according to claim 8, wherein the microorganisms on each of the bio-membranes are cultivated for 24, 48, 72, and 96 hours, respectively.

13. The method of water analysis according to claim 8, wherein the concentration of KMnO₄ is about 0.02 M (mole per liter).

14. The method of water analysis according to claim 8, wherein the microorganisms on each of the bio-membrane are stained with KMnO₄ for about 10 to 30 seconds.
